(19) United States
(12) Patent Application Publication
(10) Pub. No.: US 2024/0099964 A1
(43) Pub. Date: Mar. 28, 2024

(54) Methods and Compositions for the Prophylactic Treatment of SARS-CoV-2 virus (COVID-19)

(71) Applicant: MedinCell S.A., Jacou (FR)

(72) Inventors: Anthony Rech, Jacou (FR); Chloé Soudant, Jacou (FR); Laura Champion, Jacou (FR); Erdem Kanber, Jacou (FR); Lea Pebrel, Jacou (FR); Simon Frydrych, Jacou (FR)

(21) Appl. No.: 18/257,869

(22) PCT Filed: Dec. 16, 2021
(86) PCT No.: PCT/EP2021/086316

§ 371 Date: Jun. 15, 2023

Related U.S. Application Data
(60) Provisional application No. 63/126,125, filed on Dec. 16, 2020.

(51) Int. Cl. A61K 9/00; A61K 31/7048; A61K 47/34; A61P 31/14

(52) U.S. Cl. CPC A61K 9/0024; A61K 31/7048; A61K 47/34; A61P 31/14

(57) ABSTRACT

A method of prophylactically treating COVID-19 in subject who is at high risk for exposure to SARS-CoV-2 virus or who is been recently exposed to SARS-CoV-2, but who tests negative for the virus, by administering to said subject an effective dose of ivermectin to inhibit one or symptoms of SARS-CoV-2 virus.

### FIELD OF THE INVENTION

The present invention relates to methods of prophylactically treating SARS-CoV-2 virus (COVID-19) in a mammal with a pharmaceutical compositions comprising ivermectin, wherein the ivermectin may be in the form of a sustained release formulation comprising a triblock copolymer and a diblock copolymer.

### BACKGROUND OF THE PRESENT INVENTION

The virus SARS-CoV-2 is the causative agent of the current COVID-19 pandemic. SARS-CoV-2 is a single stranded positive sense RNA virus that is closely related to severe acute respiratory syndrome coronavirus (SARS-CoV). Studies on related viruses have suggested that IMPα/β1 may play a role during infection in signal-dependent nucleocytoplasmic shutting of the SARS-CoV nucleocapsid protein (Rowland et al. J. Virol., 79 (17) (2005), pp. 11507-11512; Timani et al., Virus Res., 114 (1-2) (2005), pp. 23-34; Wulan et al., Front. Microbiol., 6 (2015), p. 553), which may impact host cell division (Hiscox et al., J. Virol., 75 (1) (2001), pp. 506-512; Wurm et al., J. Virol., 75 (19) (2001), pp. 9345-9356). In addition, the SARS-CoV accessory protein ORF6 has been shown to antagonize the antiviral activity of the STAT1 transcription factor by sequestering IMPα/β1 on the rough ER/Golgi membrane (Frieman et al., 2007). Researchers at Monash University have proposed, based on these studies that ivermectin's nuclear transport inhibitory activity may be effective against SARS-CoV-2. (Caly et al. Antiviral Research Volume 178, June 2020, 104787).

Ivermectin is an FDA-approved broad spectrum anti-parasitic agent (Gonzalez Canga et al., AAPS J., 10 (1) (2008), pp. 42-46) that in recent years we, along with other groups, have shown to have anti-viral activity against a broad range of viruses (Gotz et al., Sci. Rep., 6 (2016), p. 23138; Lundberg et al., Antivir. Res., 100 (3) (2013), pp. 662-672; Tay et al., Antivir. Res., 99 (3) (2013), pp. 301-306; Wagstaff et al., Biochem. J., 443 (3) (2012), pp. 851-856) in vitro.

Ivermectin has been shown to be an inhibitor of the interaction between HIV-1 integrase protein (IN) and the importin (IMP) α/β1 heterodimer responsible for IN nuclear import (Wagstaff et al., J. Biomol. Screen, 16 (2) (2011), pp. 192-200). Ivermectin has also shown to inhibit infection by RNA viruses such as DENV 1-4 (Tay et al., Antivir. Res., 99 (3) (2013), pp. 301-306), West Nile Virus (Yang et al., Antivir. Res. (2020), p. 104760), Venezuelan equine encephalitis virus (VEEV) (Lundberg et al., Antivir. Res., 100 (3) (2013), pp. 662-672) and influenza (Gotz et al., Sci. Rep., 6 (2016), p. 23138). The inhibition of viral activity is believed to be from the common mechanism of RNA viruses through the action of IMPα/β1 during infection (Caly et al., Antivir. Res., 95 (2012), pp. 202-206; Jans et al., Curr. Opin. Cell Biol., 58 (2019), pp. 50-60). Ivermectin was not effective against Zika virus (ZIKV) in mice, however the authors noted that study limitations justified re-evaluation of ivermectin's against Zika virus (Ketkar et al., Diagn. Microbiol. Infect. Dis., 95 (1) (2019), pp. 38-40). In addition, in a phase III clinical trial in Thailand in 2014-2017, against DENV infection, a single daily oral dose was observed to be safe and resulted in a significant reduction in serum levels of viral NS1 protein, however no change in viremia or clinical benefit was observed (Yamasmith et al., The 34th Annual Meeting the Royal College of Physicians of Thailand, Internal Medicine and One Health, Chonburi, Thailand (2018)). Most recently Caly et al. tested the antiviral activity of ivermectin towards SARS-CoV-2, in vitro with infected Vero/hSLAM cells (Antiviral Research Volume 178, June 2020, 104787). Caly et al. reported that the results demonstrated that ivermectin has antiviral action against the SARS-CoV-2 clinical isolate in vitro, with a single dose able to control viral replication within 24-48 h and the authors concluded that, “The critical next step in further evaluation for possible benefit in COVID-19 patients will be to examine a multiple addition dosing regimen that mimics the current approved usage of ivermectin in humans. As noted, ivermectin was the focus of a recent phase III clinical trial in dengue patients in Thailand, in which a single daily dose was found to be safe but did not produce any clinical benefit.”

As of late August 2020, over 25 million people were diagnosed with COVID-19 with 845,000 deaths. In addition, it has been seen that the mortality from COVID-19 is very disparate across age groups with more than 70% of the COVID-19 deaths in the US occurring in the population aged 70 years and above (Goldstein et al. PNAS first published Aug. 20, 2020 https://doi.org/10.1073/pnas.2006392117). Assuming that a successful vaccine is identified, there will remain a population of patients who are unable to be vaccinated due to other underlying health conditions. A method of otherwise protecting populations, particularly vulnerable populations against COVID-19 is therefore desired.

The present invention is directed to methods of prophylactically treating COVID-19 by administering to a vulnerable person a suitable dose of ivermectin.

### SUMMARY OF THE INVENTION

An object of the present invention is to provide a method of prophylactically treating COVID-19 in subject who is at high risk for exposure to SARS-CoV-2 virus or who is been recently exposed to SARS-CoV-2, but who tests negative for the virus, by administering to said subject an effective dose of ivermectin to inhibit replication of the SARS-CoV-2 virus.

The present invention is further directed to a method of prophylactically treating COVID-19 in subject who is at high risk for exposure to SARS-CoV-2 virus or who is been recently exposed to SARS-CoV-2, but who tests negative for the virus, by administering to said subject an effective dose of ivermectin to inhibit replication of the SARS-CoV-2 virus, wherein the ivermectin is in sustained release formulation.

With the present method the ivermectin is incorporated into a diblock/triblock copolymer matrix.

The present invention is also directed to a method of prophylactically treating COVID-19 in subject who is at high risk for exposure to SARS-CoV-2 virus or who is been recently exposed to SARS-CoV-2, but who tests negative for the virus, by subcutaneously administering to the subject with a frequency of no more than once every 21 days:
1 mL or less of a pharmaceutical formulation comprising:
   a) ivermectin, or a pharmaceutically acceptable salt thereof, at a concentration of about 10 to 30% (w/w % of the total composition)
   b) a biodegradable triblock copolymer having the formula:

poly(lactic acid)v-poly(ethylene glycol)w,-poly(lactic acid)x
wherein v and x are the number of repeat units ranging from 24 to 682 and w is the number of repeat units ranging from 4 to 273 and v=x or v≠x;
c) a biodegradable diblock copolymer having the formula:

methoxy poly(ethylene glycol)y-poly(lactic acid)z,
wherein y and z are the number of repeat units, wherein y is the number of repeat units ranging from 3 to 45 and z is the number of units ranging from 7 to 327;
wherein the ratio of the biodegradable triblock copolymer of (b) and the biodegradable diblock copolymer of (c) is 1:3 to 1:8 or 2:1 to 6:1, in said formulation, which is insoluble in an aqueous environment.

### DETAILED DESCRIPTION OF THE INVENTION

The present invention is directed to methods and compositions for prophylactically treating COVID-19.

### Definitions

As used herein the term “biodegradable” means that the triblock and diblock copolymers will after a period of time erode or degrade in vivo to form smaller non-toxic components.

The term “parental administration” encompasses intramuscular, intraperitoneal, intra-abdominal, subcutaneous, intravenous and intraarterial. It also encompasses intradermal, intracavernous, intravitreal, intracerebral, intrathecal, epidural and intraosseous administration.

The term “about” should be considered as disclosing the range defined by the absolute values of the two endpoints. For example, the expression “from about 300 to about 400” also discloses the values 300 and 400. When used to modify a single number, the term “about” may refer to plus or minus 10% of the indicated value and includes the indicated number. For example, “about 15%” may indicate a range of 13.5% to 16.5%, and “about 1” means from 0.9 to 1.1.

The term “subject” encompasses all members of the Kingdom Animalia.

The active principle according to the invention is ivermectin and pharmaceutically acceptable salts and analogs thereof and related compounds thereto.

“SARS-CoV-2” refers to any strain of the coronavirus that causes the disease COVID-19.

The term “implant” means that once injected, the pharmaceutical composition contacts the aqueous environment and turn into solid (or semi solid) localized mass in situ. Thus, the formulations that are disclosed herein are liquids that can be easily injected through a syringe without excessive force.

The term “spatial formulations” encompass any formulations that can be applied on or into the animal body and do not necessarily have to be administered through a syringe.

As used herein “repeat units” are the fundamental recurring units of a polymer.

By “end-capped polyethylene glycol” (cPEG) refers to PEG's in which one terminal hydroxyl group is reacted and includes alkoxy-capped PEG's, urethane-capped PEG's ester-capped PEG's and like compounds. The capping group is a chemical group which does not contain a chemical function susceptible to react with cyclic esters like lactide, glycolactide, caprolactone and the like or other esters and mixtures thereof. The reaction of an end-capped PEG polymer with lactide generates a diblock cPEG-PLA copolymer.

Examples of end-capped polyethylene glycols include alkoxy capped PEG's such as methoxyPEG or ethoxyPEG, urethane-capped PEG's, ester-capped PEG's, amine-capped PEG's and amide-capped PEG's. This list of end-capped PEG's is not exhaustive and a person skilled in the art would recognize additional end-capped PEG's, which are not listed.

The abbreviation “PEG” refers to poly(ethylene glycol), poly(ethylene oxide) or poly(oxyethylene) and the terms are used interchangeably herein.

The abbreviation of “PLA” refers to polylactide, polylactic acid or poly(lactic acid) and the terms are used interchangeably herein.

The abbreviation “TB” refers to a triblock copolymer(s), while the abbreviation “DB” refers to a diblock copolymer(s).

The term “diblock” as used herein refers, for example, to an end-capped PEG-polyester copolymer. “mPEG” refers to methoxy polyethylene glycol. The PEG in the diblock copolymer may be capped with known capping entities other than a methoxy group.

The term “triblock” refers, for example, to a polyester-PEG-polyester copolymer, preferably poly(lactic acid)-PEG-poly(lactic acid) copolymer.

A “vulnerable person” is any person who is susceptible to contracting COVID-19. Of particular interest are those people who work in a high risk environment, such as health care workers and workers in crowded conditions, such as factory production lines. Other vulnerable populations include those whose health may be particularly jeopardized by contracting COVID-19, such as the elderly and those people with underlying health conditions, such as heart disease, respiratory illness, obesity, being immunocompromised etc., or those who people who cannot received an anti-SARS-CoV-2 vaccine, when available due to health issues.

“Prophylactically treating” means administering ivermectin to a subject prior to infection or becoming symptomatic with the SARS-CoV-2 virus. Prophylactically treating includes administering ivermectin to a subject who is at high risk of exposure to SARS-CoV-2 virus, who has no known exposure, in fact. Prophylactically treating also encompasses administering to a person who has a known recent exposure to SARS-CoV-2, but who is not yet testing positive for the virus. Prophylactically treating includes both wholly preventing the development of COVID-19 in a subject who has been exposed to SARS-CoV-2 virus and reducing the severity of COVID-19 in a subject who is exposed to SARS-CoV-2 virus after ivermectin administration or who has been exposed to SARS-CoV-2 virus but who is administered ivermectin prior to testing positive for the SARS-CoV-2 virus.

With the method of the invention the subject is administered a suitable dose of ivermectin prior to contracting COVID-19. It is proposed by the inventors that prophylactically administering ivermectin prior to infection with the SARS-CoV-2 virus will provide protection to the person or other mammal from the infection upon subsequent exposure or from a recent exposure because the ivermectin will prevent the development of a symptomatic case of COVID-19.

The ivermectin may be administered to the person or other mammal orally, in e.g. with a once daily dose regime. The formulation may also be administered by injection, e.g. subcutaneous injection. The ivermectin may be administered to the person or other mammal at an amount of 1-500 μg/kg, preferably 10-400 μg/kg, more preferably 50-250 μg/kg, or 100-200 μg/kg. In one embodiment the ivermectin is preferably administered at a dose of 50-150 μg/kg. The ivermectin may be administered in the form of a tablet or capsule or other suitable oral dosing form or in an injectable form.

The ivermectin may be administered at any time that there is a high risk of exposure to SARS-CoV-2. For example, the ivermectin may be administered to healthcare workers who are likely to be exposed to COVID-19 patients in the course of their work. Alternatively, the ivermectin may be administered to a vulnerable person who has been potentially exposed or may be exposed to someone with COVID-19. For example, if a resident in a care home for the elderly contract COVID-19, the other residents may be given ivermectin as a preventative measure. The ivermectin administration may be stopped when it is considered that the risk to exposure to SARS-CoV-2 has passed.

For prolonged administration of the ivermectin, the ivermectin may be administered using a sustained release formulation. The sustained release of a low dose of ivermectin may be particularly useful for individuals at continued risk for exposure to SARS-CoV-2, e.g. healthcare workers. The sustained release formulation should release the ivermectin so it results in the desired plasmatic concentration window for at least 14 days to 1 month, or at least 60 days or at least 90 days.

The sustained release formulation may be made from ivermectin incorporated into a biodegradable polymer system. Of particular use is a diblock/triblock copolymer system as described in U.S. Pat. No. 9,023,897.

Drug delivery systems such as diblock and triblock copolymers have been used to deliver a variety of drugs and are generally formulated to deliver specific drugs whether they are hydrophobic drugs or hydrophilic drugs. These drug formulations differ in polymer concentrations, types of polymers utilized, molecular weights of the polymers and solvents used in the formulations.

The type of environment in which the drug is delivered is an important consideration in formulating a drug delivery system. Thus, there exist drug delivery compositions that are prepared using temperature sensitive polymers, phase sensitive polymers, pH sensitive polymers and photosensitive polymers. See, for example, K. Al-Tahami and J. Singh “Smart Polymer Based Delivery Systems for Peptide and Proteins,” Recent Patents on Drug Delivery & Formulation, 1: pages: 65-71 Bentham Science Publishers, L T D. 2007.

U.S. Pat. No. 9,023,897 and US patent publication US2019/160171 describe pharmaceutical formulations made of biodegradable triblock and diblock polymers, which are useful for the delivery of a variety of actives.

There is a need for long-acting ivermectin formulations that provide sustained protection against the SARS-CoV-2 virus and are easier for health care professionals to prepare and administer. A long-acting formulation, which can be subcutaneously injected, and which can have a suitable dose of ivermectin in a low injection volume, with an injectable viscosity would address this need. The methods disclosed herein meet those needs and others.

The biodegradable drug delivery compositions used in the methods of the present invention are described in U.S. Pat. No. 9,023,897, the entirety of which is incorporated by reference herein.

The structure of the biodegradable triblock/diblock copolymers of the invention may also be represented as follows:
Av-Bw-Ax, which refers to the triblock copolymer poly(lactic acid)v-poly(ethylene oxide)w-poly(lactic acid)x, also identified herein as PaRb, where “a” is the PEG size in kDa and “b” is the molar ratio LA/EO (v+x/w).
Cy-Az, which refers to the diblock mPEG-PLA copolymer:methoxy-poly(ethylene glycol)y-poly(lactic acid)z, also identified herein as dPaRb, where “a” is the PEG size in kDa and “b” is the molar ratio LA/EO (z/y). The methoxy group, or other capping group, will cap one of the two hydroxyl groups of the PEG. The poly(lactic acid) chain will extend only from the free hydroxyl group.

The LA/EO ratio refers to the molar ratio of lactic acid units to ethylene oxide units that is present in the biodegradable drug delivery composition. It is determined experimentally by NMR. The LA/EO molar ratio of the triblock copolymer can range from 0.5 to 22.3. In another aspect the LA/EO molar ratio in the triblock can range from 0.5 to 6 in the pharmaceutical formulations described herein. In yet another aspect the LA/EO ratio in the triblock can range from 3 to 6.

The LA/EO ratio in the diblock can range from 0.8 to 13. In another aspect the LA/EO ratio in the diblock can range from 2 to 6 in the pharmaceutical formulations described herein. In another aspect the LA/EO ratio in the diblock can range from 2 to 4.

The degree of polymerization or DP is the number of repeat units in an average polymer chain at time t in a polymerization reaction. For example, the degree of polymerization for PEG is about 45 to 170 or it can be 4 to 273 or 3 to 45, while for PLA it can range from about 84 to 327 or it can be 24 to 682 or 7 to 327. The degree of polymerization for PEG is calculated by dividing the PEG molecular weight of the capped PEG by the EO unit molecular weight (44 Da). The DP-PLA is calculated by multiplying DP-PEG by the LA/EO ratio.

The methods of the present invention use a biodegradable drug composition comprising ivermectin and a triblock copolymer and a diblock copolymer.

The biodegradable triblock copolymer has the formula: Av-Bw-Ax, wherein A is a poly(lactic acid) and B is poly(ethylene glycol) and v and x are the number of repeat units of the poly(lactic acid) and range from 24 to 682; preferably from 40 to 50 and w is the degree of polymerization (number of repeat units) for the poly(ethylene glycol) and ranges from 4 to 273, preferably from 20 to 25, and v=x or v≠x. The triblock copolymer may be combined with a biodegradable diblock copolymer having the formula: Cy-Az, wherein A is a polyester (i.e., PLA) and C is an end-capped polyethylene glycol and y and z are the number of repeat units ranging from 7 to 371 or from 3 to 327, preferably y range from 43 to 47 and z from 129 to 141. This combination has a ratio of triblock copolymer to diblock copolymer ranging from 1:3 to 1:8 or 2:1 to 6:1 or 1:4 to 4:1. In some aspects, the ratio of triblock copolymer to diblock copolymer is 1:4 to 4:1. In some aspects, the ratio of triblock copolymer to diblock copolymer is 1:4 to 2:1. In some aspects, the ratio of triblock copolymer to diblock copolymer is 2:1 to 4:1.

Some mPEG-OH are contaminated with a small amount of OH-PEG-OH. By following the methods of the present invention and using contaminated mPEG-OH the final product would be mPEG-PLA contaminated with a small amount of PLA-PEG-PLA, which is encompassed by the present invention.

In some aspects, the present invention is directed to subcutaneous administration methods of prophylactically treating a COVID-19 in a subject. In particularly preferred embodiments, the subject is a human. In some embodiments, the subject is a human adult, who is in a high risk environment for exposure to SARS-CoV-19. In some embodiments, the subject is a vulnerable person who is over aged 70 or who is immunocompromised.

The methods of the invention comprise subcutaneous administration of a high concentration, low volume formulation of ivermectin to a subject. In some embodiments, the subcutaneous administration is to the abdomen of the subject. In other embodiments, the subcutaneous administration is to the upper arm of the subject.

Subcutaneous administration of the ivermectin formulation of the invention can result in in situ formation of solid or semi-solid implant. In these embodiments, the solid or semi-solid formulation is excisable (i.e., can be removed from the subject) following administration into the subject. A healthcare professional with skill in the art will be able to determine the preferred manner and time to excise.

In some aspects of the methods of the invention, the administration is with a frequency of no more than once every 21 days. In these aspects, the administration results in prophylactically treating of COVID-19 for at least 21 days. In some embodiments, the administration is with a frequency of no more than once every 30 days. In these aspects, the administration results in prophylactically treating of COVID-19 for at least 30 days. In other embodiments, the administration is with a frequency of no more than once every 45 days. In these aspects, the administration results in prophylactically treating of COVID-19 for at least 45 days. In other embodiments, the administration is with a frequency of no more than once every 60 days. In these aspects, the administration results in prophylactically treating of COVID-19 for at least 60 days.

According to the methods of the invention, the subject is administered 1.5 mL or less of a pharmaceutical formulation, as described herein. In some embodiments, the subject is administered 1 mL or less of the pharmaceutical formulation. In some embodiments, the subject is administered 1 mL of the pharmaceutical formulation. In other embodiments, the subject is administered 0.9 mL or less of the pharmaceutical formulation. In other embodiments, the subject is administered 0.8 mL or less of the pharmaceutical formulation. In other embodiments, the subject is administered 0.7 mL or less of the pharmaceutical formulation. In other embodiments, the subject is administered 0.6 mL or less of the pharmaceutical formulation. In other embodiments, the subject is administered 0.5 mL or less of the pharmaceutical formulation. In other embodiments, the subject is administered 0.4 mL or less of the pharmaceutical formulation. In other embodiments, the subject is administered 0.3 mL or less of the pharmaceutical formulation. In other embodiments, the subject is administered 0.2 mL or less of the pharmaceutical formulation. In other embodiments, the subject is administered 0.1 mL or less of the pharmaceutical formulation. In some embodiments, the subject is administered 0.1, 0.2, 0.3, 0.4, 0.5, 0.6, 0.7, 0.8, 0.9, or 1 mL of the pharmaceutical formulation. In some embodiments, the pharmaceutical formulation comprises 10 to 25% w/w of ivermectin.

According to the methods of the invention, the administered pharmaceutical formulations comprise ivermectin, or a pharmaceutically acceptable salt or analog thereof. Analogs of ivermectin include any 16-membered macrocyclic lactone derivatives in the avermectin family of compounds, include e.g. avermectin A₁, A₂, B₁, and B₂. (Laing et al. Trends in Parasitology Vol. 33, Issue 6, pp. 463-472(2017); Campbell W., Current Pharmaceutical Biotechnology 13(6):853-65 (2011)).

The pharmaceutically effective amount of ivermectin may vary depending on e.g. the weight and age of the subject and the risk of exposure to SARS-CoV-2, e.g. whether the potential exposure is an single event or an ongoing risk. The methods of the invention are particularly directed to formulations having the ivermectin at a concentration of at least 10-30% w/w, preferably 15-25% w/w, and more preferably 16-25% w/w. The copolymer content should be 7-20% w/w, preferably 7.5-20% w/w, more preferably 10-20% w/w, and most preferably 10-15% w/w. The triblock to diblock ratio should be in the range of 1:4 to 4:1, preferably 2:1. The delivery volume of the formulation is 1 mL or less. While there is no critical upper limit on the amount of ivermectin, the formulation should be of a viscosity suitable for injection through a syringe needle such that it can effectively prophylactically treat COVID-19 without exposing the subject to an ivermectin overdose risk and minimizing the associated side effect of ivermectin.

The length of the polyester chain is defined by its polyester to ethylene oxide molar ratio, which is between 0.5 to 22.3 or 0.5 to 6 or 3 to 6, e.g. 4 for the triblock copolymer and 0.8 to 13 or 2 to 6 or 2 to 4, e.g. 3 for the diblock copolymer.

The mass of the end-capped polyethylene glycol can range from 164 Da to 2,000 Da or may be about 2 kDa. It can range in the lower 100 to 300 Da range or in the 1 kDa to 2 kDa range.

The size of the polyethylene glycol chain ranges from 200 Da to 12 kDa in the biodegradable drug delivery composition or it can range from about 1 kDA.

The triblock copolymer is present in an amount of 2% to 19%, preferably 2.0% to 18.33% of the total weight of the composition. In another aspect, the triblock copolymer is present in an amount of 2.0% to 16.0% w/w of the total weight of the composition. In another aspect the triblock copolymer is present in an amount of 2.0% to 10.0% w/w of the total weight of the composition. In yet another aspect the triblock copolymer is present in an amount of about 8% w/w of the total weight of the composition. In yet another aspect the triblock copolymer is present in an amount of about 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5% or 10.0% w/w of the total weight of the formulation. For example, the triblock polymer may be present in an amount of about 7.10%, 7.20%, 7.30%, 7.40%, 7.50%, 7.60%, 7.70%, 7.80%, 7.90%, 8.00%, 8.10%, 8.20%, 8.30%, 8.40%, 8.50%, 8.60%, 8.70%, 8.80%, 8.90% or 9.00% w/w of the total weight of the formulation.

The diblock copolymer can be present in the biodegradable drug composition in an amount of 2.0% to 16.0% w/w of the total weight of the composition. In another aspect the diblock copolymer is present in an amount of 2.0% to 8.0% w/w of the total weight of the composition. In yet another aspect the diblock copolymer is present in an amount of about 4.0% w/w of the total weight of the composition. In yet another aspect the diblock copolymer is present in an amount of 2.0%, 2.5%, 3.0%, 3.5%, 4.0%, 4.5%, 5.0%, 5.5%, 6.0%, 6.5%, 7.0%, 7.5% or 8.0%, w/w of the total weight of the formulation. In yet another aspect the diblock copolymer is present in an amount of about 3.00%, 3.10%, 3.20%, 3.30%, 3.40%, 3.50%, 3.60%, 3.70%, 3.80%, 3.90%, 4.00%, 4.10%, 4.20%, 4.30%, 4.40%, 4.50%, 4.60%, 4.70%, 4.80%, 4.90% or 5.00% w/w of the total weight of the formulation.

The polymers are present in the pharmaceutical formulations in an amount of 7% to 28% w/w, 7.5% to 27.5% w/w, 10.0% to 20.0% w/w, or 10% to 15% w/w, or 11% to 13% w/w, of the total weight of the composition. In another aspect the total weight of the polymers present in the biodegradable drug composition is 10.0% to 15.0% w/w of the total weight of the composition. In yet another aspect the polymers are present in the biodegradable drug composition at about 12.5% w/w of the total weight of the composition. In yet another aspect the polymers are present in the biodegradable drug formulation at about 10.0%, 10.5%, 11.0%, 11.5%, 12.0%, 12.5%, 13.0%, 13.5%, 14.0%, 14.5% or 15.0% w/w of the total weight of the formulation. In yet another aspect the polymers are present in an amount of about 11.50%, 11.60%, 11.70%, 11.80%, 11.90%, 12.00%, 12.10%, 12.20%, 12.30%, 12.40%, 12.50%, 12.60%, 12.70%, 12.80%, 12.90%, 13.00%, 13.10%, 13.20%, 13.30%, 13.40% or 13.50% w/w of the total weight of the formulation.

The ratio of the biodegradable triblock copolymer (b) and the biodegradable diblock copolymer (c) is 1:3 to 1:8 or 2:1 to 6:1 or 1:4 to 4:1 in the pharmaceutical formulations of the invention.

In one embodiment, the ratio of the biodegradable triblock copolymer and the biodegradable diblock copolymer is selected from 1:2, 1:4, 2:1 and 4:1.

In some embodiments, the ratio of the biodegradable triblock copolymer of and the biodegradable diblock copolymer is 4:1. In other embodiments, the ratio of the biodegradable triblock copolymer of and the biodegradable diblock copolymer is 1:4. In other embodiments, the ratio of the biodegradable triblock copolymer of and the biodegradable diblock copolymer is 2:1.

The pharmaceutical formulations used in the methods of the disclosure can further comprise a pharmaceutically acceptable carrier, adjuvant or vehicle. An acceptable carrier can be saline, buffered saline and the like. The adjuvant can be formulated simultaneously when mixing the drug. In this regard the adjuvants that can be used are alum, aluminum phosphate, calcium phosphate, MPL™, CpG motifs, modified toxins, saponins, endogenous stimulatory adjuvants such as cytokines, Freunds complete and incomplete adjuvants, ISCOM type adjuvants, muramyl peptides and the like.

The pharmaceutical formulations used in the methods of the invention also include an organic solvent. In preferred embodiments, the organic solvent is a water-soluble organic solvent. The organic solvent that can be used in the methods described herein are selected from the group of: benzyl alcohol, benzyl benzoate, diethylene glycol dimethyl ether (Diglyme), diethylene glycol monoethyl ether (DEGMEE), dimethyl isosorbide (DMI), dimethyl sulfoxide (DMSO), ethyl acetate, ethyl benzoate, ethyl lactate, ethylene glycol monoethyl ether acetate, glycerol formal, methyl ethyl ketone, methyl isobutyl ketone, N-ethyl-2-pyrrolidone, N-methyl-2-pyrrolidinone (NMP), pyrrolidone-2, tetraglycol, triacetin, tributyrin, tripropionin (tripro), or triethylene glycol dimethyl ether (triglyme) and mixtures thereof. A preferred organic solvent is the water soluble organic solvent, DMSO.

The organic solvent is present in an amount of 52% to 80% w/w or 52.5% to 80% w/w or 60% to 75% w/w or 65% to 70% w/w or 62% to 72% w/w of the total composition. In another aspect the organic solvent used in the preparation of the biodegradable drug delivery composition is present in an amount of 62% to 72% w/w of the total composition. In yet another aspect the solvent used in the preparation of the biodegradable drug delivery composition is present in an amount of 65% to 70% w/w of the total composition.

In some embodiments, the organic solvent is DMSO. Triglycerides such as triacetin or tripropionin may also be included with the DMSO. The amount of DMSO that can be used in the pharmaceutical formulations of the methods of the present invention can be from 52% to 80% w/w 52.5% to 80% w/w, preferably from 60% to 75% w/w, more preferably from 62% to 72% w/w.

In the biodegradable drug delivery composition, also referenced herein as a pharmaceutical formulation, of the present invention, the amount of ivermectin is released gradually over an extended period of time. This slow release can be continuous or discontinuous, linear or non-linear and can vary due to the composition of the triblock copolymer and diblock copolymer.

In one aspect, the biodegradable drug delivery composition can deliver the ivermectin for at least 21 days. In one aspect, the biodegradable drug delivery composition can deliver the ivermectin for 21 days, 30 day, 45 days, 60 days, 90 days, 120 days, or 180 days. In another aspect, the biodegradable drug delivery composition can deliver the ivermectin for at about 21 to 30 days or at about 28 to 31 days. In another aspect, the biodegradable drug delivery composition can deliver the ivermectin for at least 30 days. In another aspect, the biodegradable drug delivery composition can deliver the ivermectin for at about 56 to 63 days. In another aspect, the biodegradable drug delivery composition can deliver the ivermectin for at least 60 days. In one aspect, the biodegradable drug delivery composition can deliver the ivermectin for at least 90 days.

In the methods of the present invention, the administration results in an effective amount of ivermectin being released from the formulation to prophylactically treat the development of COVID-19 following exposure to SARS-CoV-2 virus for an extended period of time. In some embodiments, the administration is in prophylactically treat the development of COVID-19 symptoms following exposure to SARS-CoV-2 virus for a duration of 21 days to 90 days. In some embodiments, the administration is effective in prophylactically treat the development of COVID-19 following exposure to SARS-CoV-2 virus for 30 days to 90 days. In other embodiments, the administration is effective in prophylactically treat the development of COVID-19 following exposure to SARS-CoV-2 virus for 30 days to 60 days. In some embodiments, the administration is effective in prophylactically treat the development of COVID-19 following exposure to SARS-CoV-2 virus for 30 days. In other embodiments, the administration is effective in prophylactically treat the development of COVID-19 following exposure to SARS-CoV-2 virus for 45 days. In other embodiments, the administration is effective in prophylactically treat the development of COVID-19 following exposure to SARS-CoV-2 virus for 60 days. In other embodiments, the administration is effective in prophylactically treat the development of COVID-19 following exposure to SARS-CoV-2 virus for 90 days.

In some aspects of the methods of the invention, the release of ivermectin from the pharmaceutical formulation is such that therapeutically effective levels of ivermectin are achieved within 24 hours, 48 hours, or 72 hours, of subcutaneous administration. With therapeutically effective levels of ivermectin achieved within 24 hours, 48 hours, or 72 hours, of subcutaneous administration, alternative, immediate release ivermectin formulations (for example, immediate release oral formulations or immediate release injectable formulations) are not required to ensure adequate ivermectin levels in a subject. That is, a “loading dose” or supplemental oral dose of ivermectin is not required in the methods of the invention. Thus, in some embodiments, the methods are implemented in the absence of a loading dose or supplemental oral ivermectin. This is particularly the case if there is no known exposure to the SARS-CoV-2 virus. Alternatively, if an exposure to the SARS-CoV-2 virus is known or suspected, an immediate loading oral dose of ivermectin may be given in conjunction with the subcutaneous administration of the sustained release composition.

Using the methods of the invention, a therapeutically effective amount of the ivermectin will have been released by a target date. Thus, with an amount of a “30-day formulation,” about 80%, of the ivermectin will have been cumulatively released by 30 days post administration. The term “cumulatively released” as used herein, refers to the total amount of ivermectin (by weight) released by a particular point in time, as a percentage of the total amount of ivermectin in the formulation. Cumulative release can be measured by, for example, the in vitro release (IVR) methods known in the art and described herein.

In some embodiments, less than about 50% of the ivermectin in the formulation is cumulatively released at 24 hours post administration. In other embodiments, about 20% to about 45% of the ivermectin in the formulation is cumulatively released at 24 hours post administration.

In some embodiments, about 80% of the ivermectin in the formulation is cumulatively released at 30 days post administration.

The pharmaceutical formulations used in the methods of the disclosure are injectable liquids at room temperature and can be injected through a syringe without excessive force. The compositions are also in situ forming and biodegradable and turn into solid or soli solid implants when injected into the animal.

In some aspects of the methods of the invention, the pharmaceutical formulation is administered from a pre-filled syringe (PFS). A PFS is a syringe in which contains an appropriate amount of the pharmaceutical formulation and is ready for subcutaneous administration, preferably by a healthcare professional. In some embodiments of the methods of the invention, the pharmaceutical formulation is administered from a single pre-filled syringe. In other embodiments, the pharmaceutical formulation is administered from more than one pre-filled syringe, for example, from 2, 3, 4, 5, or 6 or more pre-filled syringes.

According to the disclosure, the volume of the pharmaceutical formulation in the pre-filled syringe is 1.5 ml or less. In some embodiments, the volume of the pharmaceutical formulation in the pre-filled syringe is 1.0 ml or less. In some embodiments, the volume of the pharmaceutical formulation in the pre-filled syringe is between 0.1 mL and 0.9 mL. In some embodiments, the volume of the pharmaceutical formulation in the pre-filled syringe is between 0.1 mL and 0.8 mL. In some embodiments, the volume of the pharmaceutical formulation in the pre-filled syringe is between 0.1 mL and 0.5 mL. In some embodiments, the volume of the pharmaceutical formulation in the pre-filled syringe is about 0.1 mL. In some embodiments, the volume of the pharmaceutical formulation in the pre-filled syringe is about 0.2 mL. In some embodiments, the volume of the pharmaceutical formulation in the pre-filled syringe is about 0.3 mL. In some embodiments, the volume of the pharmaceutical formulation in the pre-filled syringe is about 0.4 mL. In some embodiments, the volume of the pharmaceutical formulation in the pre-filled syringe is about 0.5 mL. In some embodiments, the volume of the pharmaceutical formulation in the pre-filled syringe is about 0.6 mL. In some embodiments, the volume of the pharmaceutical formulation in the pre-filled syringe is about 0.7 mL. In some embodiments, the volume of the pharmaceutical formulation in the pre-filled syringe is about 0.8 mL. In some embodiments, the volume of the pharmaceutical formulation in the pre-filled syringe is about 0.9 mL. In some embodiments, the volume of the pharmaceutical formulation in the pre-filled syringe is about 1.0 mL.

Methods for preparing the pharmaceutical formulations used in the methods of the invention are disclosed in, for example U.S. Pat. No. 9,023,897, incorporated by reference herein.

For the foregoing embodiments, each embodiment disclosed herein is contemplated as being applicable to each of the other disclosed embodiments. For instance, the elements recited in the method embodiments can be used in the pharmaceutical composition or formulation embodiments described herein and vice versa.

The following examples are for illustrative purposes, and are intended to be nonlimiting. Those of skill in the art will readily recognize a variety of features which can be changed or modified to yield essentially the same results.

### EXAMPLES

### Example 1

### Formulation Preparation

Typically, ivermectin formulations are prepared as detailed below:
In a 10 mL glass vial, the required amount of DS (Drug Substance) was weighed,
DMSO was added on top of the DS, using a 5 mL syringe equipped with a 16 G 1½″ needle or a glass Pasteur pipette
The mixture was left on a roller mixer until complete solubilization of the DS.

After complete dissolution of the DS:
Triblock polymer was added to the vial,
Diblock polymer was added to the vial,
The vial was flushed with nitrogen
The vial was placed on a roller mixer, allowing the polymers to fully dissolve in the DMSO at room temperature (RT) overnight.

### Example 2

IVR (In Vitro Release) Test 1

Typically, ivermectin dose was fixed at 120 mg or 45 mg and depending on the concentration of IVM in the formulation, the depot mass and volume was adjusted to reach the chosen dose.

The following steps were performed for the IVR with 125 mg dose depots:
100 mL of PBS 1×+0.3% Brij35® were added in a 250 mL Erlenmeyer flasks,
A 19 G 1″ and a 25 G ⅝″ needle was inserted (to relieve pressure) into the vial containing the formulation,
A syringe was added to the 19 G needle and filled with the required amount of drug product,
The syringe was disconnected from the needle and the piston moved back and forth several times to remove air bubbles,
A 21 G ⅝″ needle was attached to the syringe,
The formulation was injected into a gelatin capsule size #000,
Using tweezers, the capsule was placed directly into an Erlenmeyer containing 100 mL of buffer,
The Erlenmeyer flask was placed into a 37° C. climatic chamber on an orbital agitator set at 93 rpm.

At the predetermined time points (2, 4, 8 hours, 1, 2, 3, 6, 8, 10, 14, 17 and 21 days after depot formation), 2.5 mL of released buffer were withdrawn from the Erlenmeyer flasks and filtered through a 0.22 μm cellulose filter into a HPLC glass vial and a back-up Eppendorf. Remaining buffer was discarded and refreshed with 100 mL of PBS 1×+0.3% Brij35®.

Samples were analyzed by ultra-performance Liquide Chromatography (UPLC) using method described in table 1.

TABLE 1
UPLC method for IVM quantification in IVR medium

| Column | Acquity UPLC HSS T3 C18 1.8 μm (2.1 × 50 mm) | | |
|---|---|---|---|
| Column temperature | 35° C. | | |
| Flow rate | 0.3 mL/min | | |
| Injection Volume | 1 μL | | |
| Detection | UV at 244 nm | | |
| Sampling rate | 5 points/s | | |
| Aqueous eluant A | Ultrapure water:Formic acid (100:0.1, v/V) | | |
| Organic eluant B | Acetonitrile:Formic acid (100:0.1, v/V) | | |
| Elution program | Time (min) | Eluant A (%) | Eluant B (%) |
| | 0.0 | 20 | 80 |
| | 4.0 | 13 | 87 |
| | 4.5 | 10 | 90 |
| | 5.0 | 20 | 80 |
| | 8.5 | 20 | 80 |

| | |
|---|---|
| Analysis run time | 8.5 min |
| Retention Time | 3.4 and 4.3 minutes |
| (RT) of Ivermectin | |
| (H2B1b and H2B1a) | |

Table 2 summarizes the composition tested in IVR tests.

TABLE 2

| | IVM Drug | | | |
|---|---|---|---|---|
| | Substance | TB P1R4 | DB dP2R3 | DMSO |
| | (% w/w) | (% w/w) | (% w/w) | (% w/w) |
| F01 | 20.0 | 8.0 | 2.0 | 70.0 |
| F02 | 20.0 | 2.0 | 8.0 | 70.0 |
| F03 | 10.0 | 4.0 | 16.0 | 70.0 |
| F04 | 10.0 | 16.0 | 4.0 | 70.0 |
| F05 | 10.0 | 8.0 | 2.0 | 80.0 |
| F06 | 15.0 | 10.0 | 5.0 | 70.0 |
| F07 | 20.0 | 16.0 | 4.0 | 60.0 |
| F08 | 20.0 | 4.0 | 16.0 | 60.0 |
| F09 | 10.0 | 2.0 | 8.0 | 80.0 |
| F10 | 15.0 | 8.3 | 4.2 | 72.5 |
| F11 | 15.0 | 13.3 | 6.7 | 65.0 |
| F12 | 20.0 | 10.0 | 5.0 | 65.0 |
| F13 | 17.5 | 8.3 | 4.2 | 70.0 |
| F14 | 17.5 | 10.0 | 5.0 | 67.5 |
| F15 | 12.5 | 11.7 | 5.8 | 70.0 |
| F16 | 20.0 | 6.7 | 3.3 | 70.0 |
| F17 | 15.0 | 11.7 | 5.8 | 67.5 |
| F18 | 10.0 | 10.0 | 5.0 | 75.0 |
| F19 | 10.0 | 13.3 | 6.7 | 70.0 |
| F20 | 12.5 | 10.0 | 5.0 | 72.5 |
| F21 | 15.0 | 6.7 | 3.3 | 75.0 |

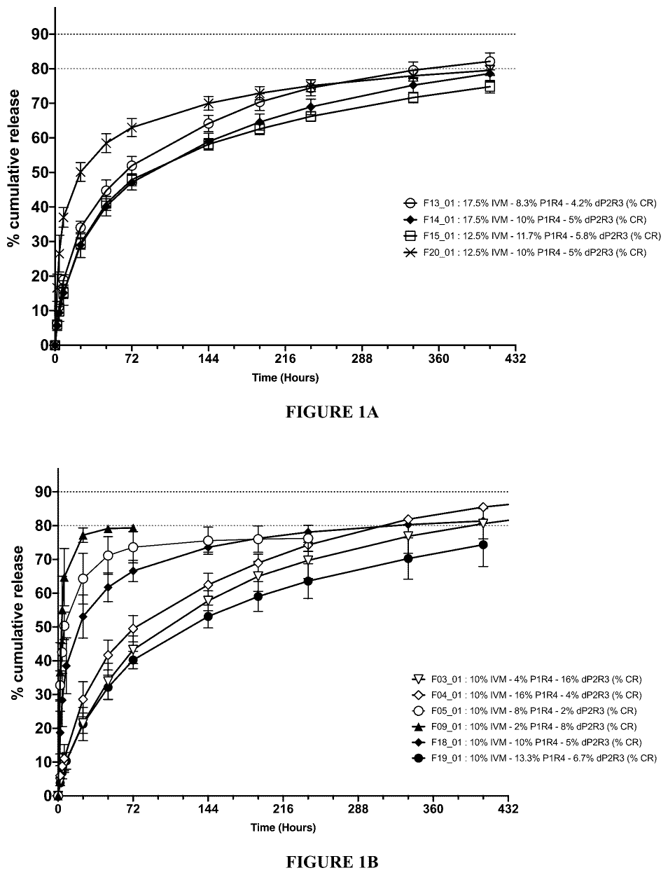

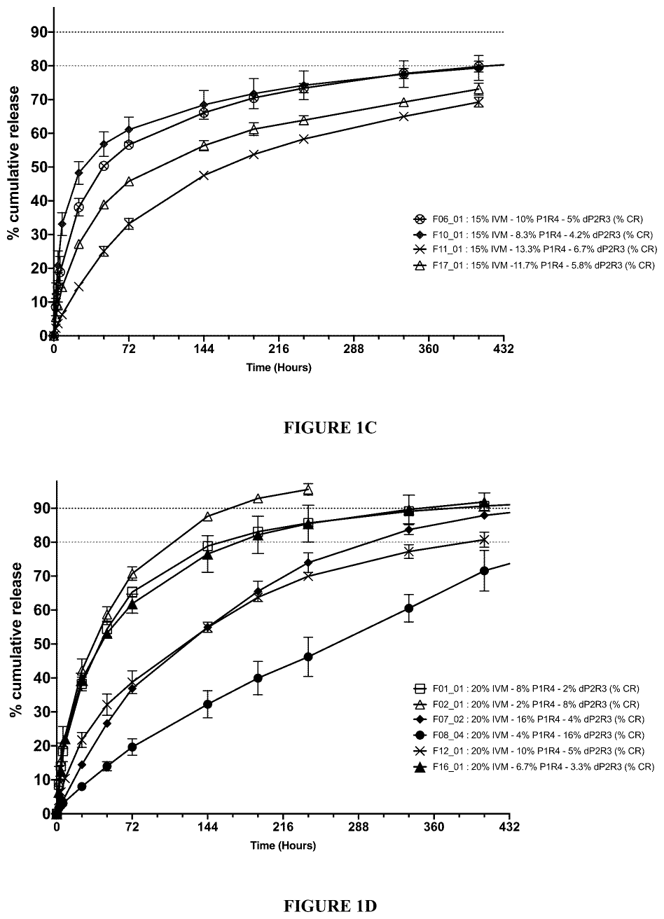

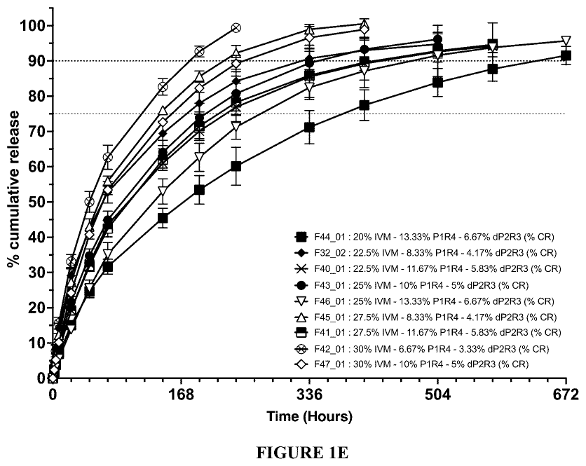

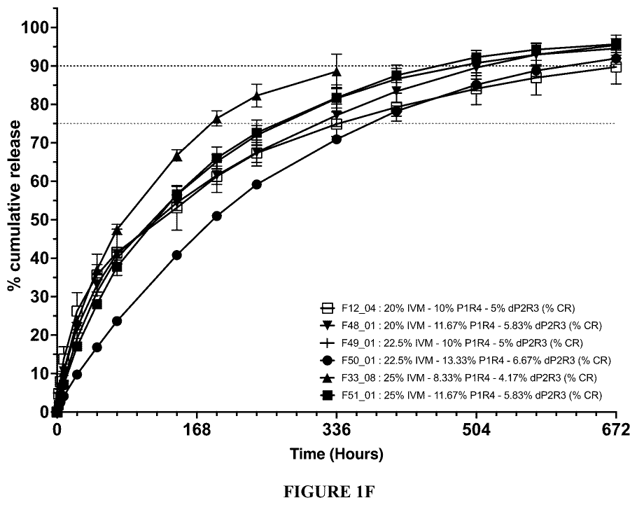

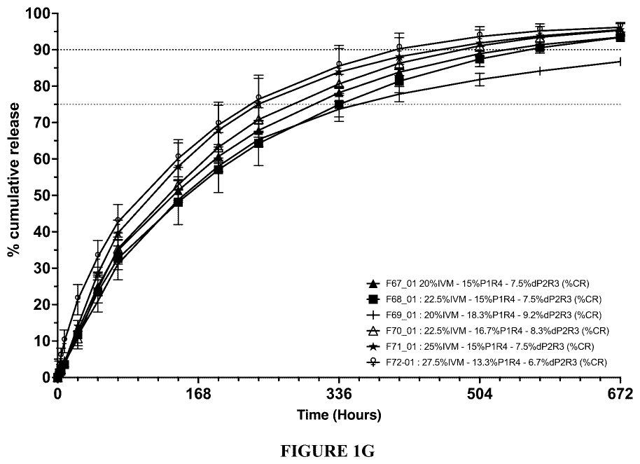

The release profiles of the IVR (in Vitro Release) tests are presented in FIG. 1A through FIG. 1D.

Release Targeted:
7D<NLT 75%<14D

From the in vitro release profile it was seen that IVR profiles obtained can be widely controlled by changing the P1 R4/dP2R3 copolymers relative ratio as well as the total copolymer content in the formulation. A minimum of 20% of (IVM+Copolymers) formed a robust depot which releases more than 3 days and a maximum of 40% of (IVM+Copolymers) had an acceptable IVM release profile.

IVR (In Vitro Release) Test 2

The ivermectin drug substance dose was fixed at 150 mg for this In Vitro Release test. Depending on the concentration of IVM in the formulation, the depot mass and volume were adjusted to reach the chosen dose.

The following steps were performed for the IVR with 150 mg dose depots:
100 mL of PBS 1×+0.3% Brij35® were added in a 250 mL Erlenmeyer flasks,
A 19 G 1″ and a 25 G ⅝″ needle was inserted (to relieve pressure) into the vial containing the formulation,
A syringe was added to the 19 G needle and filled with the required amount of drug product,
The syringe was disconnected from the needle and the piston moved back and forth several times to remove air bubbles,
A 21 G ⅝″ needle was attached to the syringe,
The formulation was injected into a gelatin capsule size #000,
Using tweezers, the capsule was placed directly into an Erlenmeyer containing 100 mL of buffer,
The Erlenmeyer flask was placed into a 37° C. climatic chamber on an orbital agitator set at 93 rpm.

At the predetermined time points (2, 4, 8 hours, 1, 2, 3, 6, 8, 10, 14, 17, 21, 24 and 28 days after depot formation), 2.5 mL of release buffer was withdrawn from the Erlenmeyer flasks and filtered through a 0.22 μm cellulose filter into a HPLC glass vial and a back-up Eppendorf. Remaining

Release buffer was discarded and refreshed with 100 mL of PBS 1×+0.3% Brij35. Samples were analyzed by ultra-performance Liquid Chromatography (UPLC) using method described in table 1.

Table 2a summarizes the formulation compositions tested via IVR analysis.

TABLE 3a

| | IVM Drug | Triblock | Diblock | Total | |
|---|---|---|---|---|---|
| | substance | P1R4 | dP2R3 | Copolymer | DMSO |
| | (% w/w) | (% w/w) | (% w/w) | (% w/w) | (% w/w) |
| F40 | 22.50 | 11.67 | 5.83 | 17.50 | 60.00 |
| F41 | 27.50 | 11.67 | 5.83 | 17.50 | 55.00 |
| F42 | 30.00 | 6.67 | 3.33 | 10.00 | 60.00 |
| F43 | 25.00 | 10.00 | 5.00 | 15.00 | 60.00 |
| F44 | 20.00 | 13.33 | 6.67 | 20.00 | 60.00 |
| F45 | 27.50 | 8.33 | 4.17 | 12.50 | 60.00 |
| F46 | 25.00 | 13.33 | 6.67 | 20.00 | 55.00 |
| F47 | 30.00 | 10.00 | 5.00 | 15.00 | 55.00 |
| F48 | 20.00 | 11.67 | 5.83 | 17.50 | 62.50 |
| F49 | 22.50 | 10.00 | 5.00 | 15.00 | 62.50 |
| F50 | 22.50 | 13.33 | 6.67 | 20.00 | 57.50 |
| F51 | 25.00 | 11.67 | 5.83 | 17.50 | 57.50 |
| F67 | 20.00 | 15.00 | 7.50 | 22.50 | 57.50 |
| F68 | 22.50 | 15.00 | 7.50 | 22.50 | 55.00 |
| F69 | 20.00 | 18.33 | 9.17 | 27.50 | 52.50 |
| F70 | 22.50 | 16.67 | 8.33 | 25.00 | 52.50 |
| F71 | 25.00 | 15.00 | 7.50 | 22.50 | 52.50 |
| F72 | 27.50 | 13.33 | 6.67 | 20.00 | 52.50 |

The release profiles of the In Vitro Release tests are presented in FIG. 1E through FIG. 1G.

The release targeted is:
10D<NLT 75%<30D

From the in vitro release profile, it was seen that the obtained profiles can be widely controlled by changing the IVM and total copolymer content in the formulation. A minimum of 37.5% of (IVM+Copolymers) formed robust depot which control the release in the first 24 hours and a maximum of 47.5% of (IVM+Copolymers) had an acceptable extended IVM release profile.

### Example 3

Pharmacokinetics Study on Dogs

Based on the results obtained in the in vitro studies, formulations were selected for pharmacokinetics (PK) study.

Six test items were selected for the PK 1 study in dogs: F01, F02, F06, F12, F13 and F14. The target IVM dose was set at 4.2 mg/kg and injected formulation volumes were adjusted depending on the API loading of tested formulations and on the animal body weight. Formulations were administered subcutaneously at a constant rate in the inter-scapular area of dogs using a 1 mL BBraun Omnifix®-F Luer Lock syringe with a 23 G ⅝″ needle. Actual dose of ivermectin was calculated as follow:

$$actual dose ( mg / kg ) = \frac{\begin{matrix}\begin{matrix}( weight syringe before injection ( mg ) - \\ weight syringe after i njection ( mg ) ) *\end{matrix} \\ actual IVM concentration\end{matrix}}{Weight dog ( kg )}$$

Each group was composed of 4 animals.

Around 1.2 mL of blood samples were collected using a single used sterile needle connected to a plastic syringe or vacutainer containing K2-EDTA anticoagulant before and after injection at different time points: −1 day (D), 0.5 h, 0.75 h, 1 h, 2 h, 4 h, 6 h, 10 h, 24 h, 2 D, 3D, 4D, 5D, 7D, 10D, 14D, 17D, 19D, 21 D, 25 D and 28 D. Blood samples were centrifuged and the plasma from each time point was retained. The IVM content was quantified in each plasma sample using appropriate LC/MS-MS method.

Table 3 details the compositions of formulations tested in vivo during the first PK study.

TABLE 4

| | IVM Drug | | | |
|---|---|---|---|---|
| | Substance | TB P1R4 | DB dP2R3 | DMSO |
| | (% w/w) | (% w/w) | (% w/w) | (% w/w) |
| F01 | 20.00 | 8.00 | 2.00 | 70.00 |
| F02 | 20.00 | 2.00 | 8.00 | 70.00 |
| F06 | 15.00 | 10.00 | 5.00 | 70.00 |
| F12 | 20.00 | 10.00 | 5.00 | 65.00 |
| F13 | 17.50 | 8.33 | 4.17 | 70.00 |
| F14 | 17.50 | 10.00 | 5.00 | 67.5 |

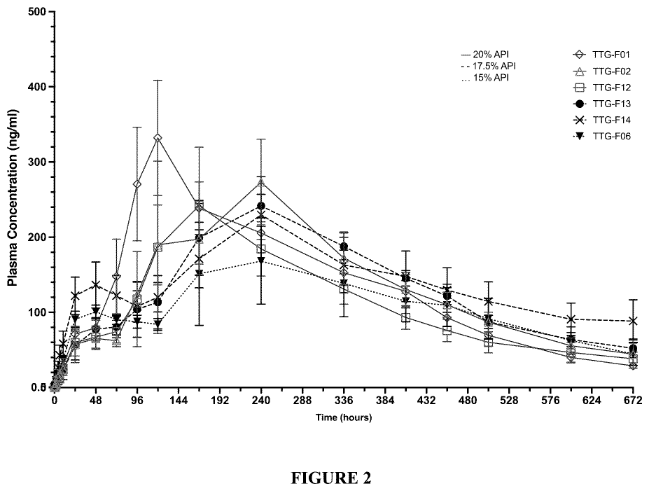

FIG. 2. presents the release profiles for the PK1 formulations.

### Example 4

Pharmacokinetics Study on Dogs

Three test items were selected for the PK 2 study in dogs: F13, F33 and F37. Two IVM doses were evaluated: 1.9 and 3.8 mg/kg. Injected formulation volumes were adjusted depending on the API loading of tested formulations and on the animal body weight. Formulations were administered subcutaneously at a constant rate in the inter-scapular area of dogs using a 1 mL Henke-Ject Luer Lock syringe with a 23 G ⅝″ needle.

Actual dose of ivermectin was calculated as follow:

$$actual dose ( mg / kg ) = \frac{\begin{matrix}\begin{matrix}( weight syringe before injection ( mg ) - \\ weight syringe after i njection ( mg ) ) *\end{matrix} \\ actual IVM concentration\end{matrix}}{Weight dog ( kg )}$$

Each group is composed of 4 animals.

Around 1.2 mL of blood samples are collected using a single used sterile needle connected to a plastic syringe or vacutainer containing K2-EDTA anticoagulant before and after injection at different time points: −4 days (D), 1 h, 2 h, 4 h, 6 h, 10 h, 24 h, 2 D, 3 D, 4 D, 5 D, 6 D, 7 D, 8 D, 9 D, 10 D, 14 D, 17 D, 19 D, 21 D, 25 D and 28 D. Additional blood samples are collected at 35 D, 42 D, 49 D and 56 D on some animals. Blood samples are centrifuged and the plasma from each time point is retained. The IVM content is quantified in each plasma sample using appropriate LC/MS-MS method.

Table 4 details the compositions of formulations tested in vivo during the second PK study.

TABLE 5

| | IVM Drug | | | |
|---|---|---|---|---|
| | Substance | TB P1R4 | DB dP2R3 | DMSO |
| | (% w/w) | (% w/w) | (% w/w) | (% w/w) |
| F13 | 17.50 | 8.33 | 4.17 | 70.00 |
| F33 | 25.00 | 8.33 | 4.17 | 62.50 |
| F37 | 16.25 | 8.33 | 4.17 | 71.25 |

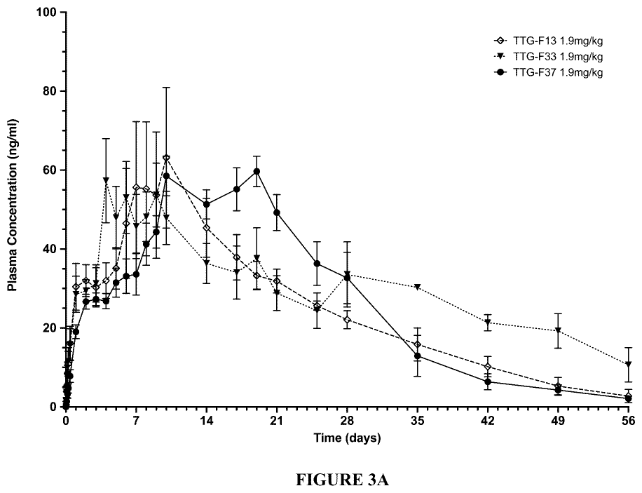

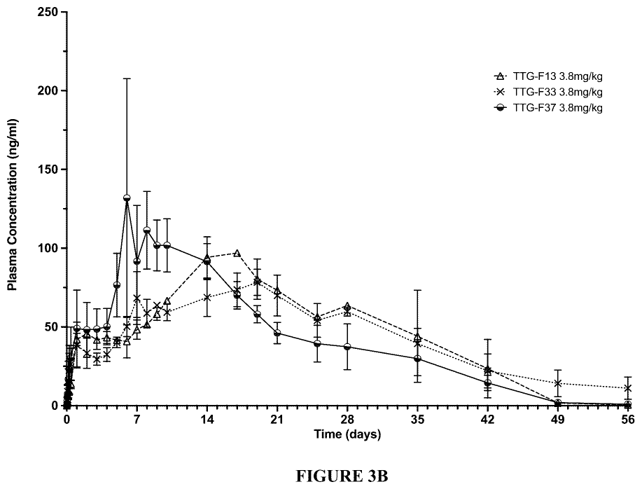

FIGS. 3A and 3B present the release profiles for the PK2 formulations at the doses of 1.9 and 3.8 mg/kg respectively.

### Example 5

Pharmacokinetics Study on Dogs

Six test items were selected for the PK 3 study in dogs: F33, F43, F49, F50, F51. The target IVM dose was set at 3.8 mg/kg. Injected formulation volumes were adjusted depending on the API loading of tested formulations and the animal's body weight. Formulations were administered subcutaneously at a constant rate in the inter-scapular area of dogs using a 1 mL Henke-Ject® Luer Lock syringe with a 23 G ⅝″ needle.

Each group was composed of 5 male animals.

Around 1.2 mL of blood samples were collected, using a single-use sterile needle connected to a plastic syringe or vacutainer containing K2-EDTA anticoagulant before and after injection at different time points: −1 day (D), 1 h, 2 h, 4 h, 6 h, 10 h, 24 h, 2 D, 3D, 4 D, 5 D, 7 D, 10 D, 14 D, 17 D, 19 D, 21 D, 25 D, 28 D, 35 D, 42 D, 49 D, 56 D, 63 D, 70 D, 77 D, 84 D, 91 D, 98 D, 105 D, 112 D, 119 D. Blood samples were centrifuged and the plasma from each time point was retained. The IVM content was quantified in each plasma sample using an appropriate LC/MS-MS method.

Table 5 details the compositions of formulations tested in vivo during this PK study.

TABLE 5

| | IVM Drug | Triblock | Diblock | |
|---|---|---|---|---|
| | substance | P1R4 | dP2R3 | DMSO |
| | (% w/w) | (% w/w) | (% w/w) | (% w/w) |
| F33 | 25.00 | 8.33 | 4.17 | 62.50 |
| F43 | 25.00 | 10.00 | 5.00 | 60.00 |
| F49 | 22.50 | 10.00 | 5.00 | 62.50 |
| F50 | 22.50 | 13.33 | 6.67 | 57.50 |
| F51 | 25.00 | 11.67 | 5.83 | 57.50 |

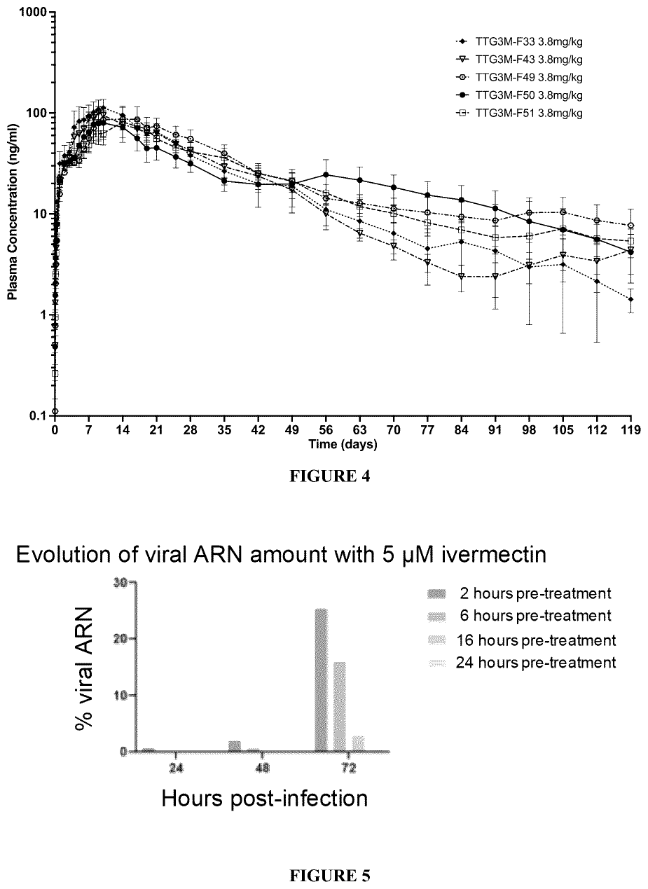

FIG. 4. presents the release profiles for the PK3 formulations.

### Example 6

Assessment of Ivermectin effect on preventing SARS-CoV-2 infection Vero-E6 cells were incubated with 0.01, 0.025, 0.05, 0.1, 0.5, 1 or 5 μM concentrations of ivermectin during 2, 6, 16 or 24 hours. Then, cells were infected with SARS-CoV-2 at a multiplicity of infection (MOI) of 0.001, 0.01 or 0.1. After 2 hours of incubation in the presence of the virus, medium with the same concentration of ivermectin was added again. After 24, 48 or 72 hours, viral charge is quantified by qRT-PCR.

For avoiding false positives, cytotoxicity was evaluated by culturing non-infected cells in the same conditions and with the same concentrations of ivermectin. Cell viability was then determined.

No cell toxicity was observed for any the concentrations tested. RT-qPCR results show that viral replication is highly inhibited when cellules were pretreated with ivermectin at a concentration of 5 μM. At 72 hours post-infection, a clear preincubation-time dependency is observed: the longer the preincubation time with ivermectin, the higher the inhibition of viral replication.

FIG. 5 presents typical results that may be obtained with the 5 μM concentrations.

### Example 7

Pharmacokinetics/Pharmacodynamics (PK/PD) Study on Hamsters Infected with SARS-CoV2 Virus

At day X (to be determined based on a previous PK study on hamsters), one ivermectin formulation at a bodyweight concentration to be determined is injected subcutaneously in hamsters (n=5). At day 0, a number of SARS-CoV2 virus particles to be determined are administered by aerosol in nasal cavity in each hamster. Plasma samples as well as lungs, liver, plasma, spleen, trachea and nasal turbinate are taken at days 0, 2, 4, 7 and 14. The viral charge is determined by RT-PCR. Histopathology is performed in the extracted organs. Immune response is evaluated. Symptoms are followed during the full duration of the study. Results are compared to those obtained from non-treated animals.

1. A method of prophylactically treating COVID-19 in subject who is at high risk for exposure to SARS-CoV-2 virus or who has been recently exposed to SARS-CoV-2, but who tests negative for the virus, which comprises administering to said subject an effective dose of ivermectin to inhibit development of one or more COVID-19 symptoms.

2. A method of prophylactically treating COVID-19 in subject who is at high risk for exposure to SARS-CoV-2 virus or who has been recently exposed to SARS-CoV-2, but who tests negative for the virus, which comprises administering to said subject an effective dose of ivermectin to inhibit development of one or more COVID-19 symptoms, wherein the ivermectin is in sustained release formulation.

3. The method of claim 2, wherein the ivermectin is incorporated into a matrix comprising a diblock copolymer and a triblock copolymer, such as a PEG-PLA/PLA-PEG-PLA copolymer matrix.

4. A method of prophylactically treating COVID-19 in a subject who is at high risk for exposure to SARS-CoV-2 virus or who has been recently exposed to SARS-CoV-2, but who tests negative for the virus, which comprises subcutaneously administering to the subject with a frequency of no more than once every 21 days:
1.5 mL or less, preferably 1 mL or less, of a pharmaceutical formulation comprising:
a) ivermectin, or a pharmaceutically acceptable salt thereof, at a concentration of about 10% w/w to 30% w/w of the total weight of the formulation;
b) a biodegradable triblock copolymer having the formula:
poly(lactic acid)v-poly(ethylene glycol)w,-poly(lactic acid)x
wherein v and x are numbers of repeat units ranging from 24 to 682 and w is a number of repeat units ranging from 4 to 273 and v=x or v≠x;
c) a biodegradable diblock copolymer having the formula:
methoxy poly(ethylene glycol)y-poly(lactic acid)z,
wherein y and z are numbers of repeat units, wherein y is a number of repeat units ranging from 3 to 45 and z is a number of units ranging from 7 to 327;
wherein a ratio of the biodegradable triblock copolymer of (b) and the biodegradable diblock copolymer of (c) is 1:3 to 1:8 or 2:1 to 6:1, or 2:1, in said formulation, which is insoluble in an aqueous environment.

5. The method of claim 2, wherein the formulation further comprises an organic solvent, preferably DMSO.

6. The method of in claim 2, wherein the concentration of the ivermectin is 16% w/w to 25% w/w of the total weight of formulation.

7. The method of claim 4, wherein the biodegradable triblock is P1 R4 with w being about 20-25, and v and x each independently being about 40-50, the biodegradable diblock is dP2R3, with y being about 43-47 and z being about 129-141 and wherein a ratio of P1R4 and dP2R3 is 1:4 to 4:1, preferably 2:1.

8. The method of claim 3, wherein the triblock copolymer is present in an amount of about 2% to 19%, preferably about 2% to 18.33%, more preferably about 2% to 16% w/w of the total weight of the formulation.

9. The method of claim 8, wherein the triblock copolymer is present in an amount of about 2% w/w to 10% w/w of the total weight of the formulation, preferably about 8% w/w of the total weight of formulation.

10. The method of claim 3, wherein the diblock copolymer is present in an amount of about 2% w/w to 16% w/w of the total weight of the formulation.

11. The method of claim 10, wherein the diblock copolymer is present in an amount of about 2% w/w to 8% w/w of the total weight of the formulation, preferably about 4% w/w of the total weight of formulation.

12. The method of claim 3, wherein the triblock and diblock copolymers are present in a total amount of about 7% to about 28%, preferably about 7.5% to about 27.5%, more preferably about 10% to about 20% w/w of the total weight of the formulation.

13. The method of claim 12, wherein the triblock and diblock copolymers are present in a total amount of about 10% w/w to 15% w/w of the total weight of the formulation, preferably about 12% w/w of the total weight of the formulation.

14. The method of claim 5, wherein the DMSO is present in an amount of about 52% to about 80%, preferably about 52.5% to about 80%, more preferably about 62% to about 72% (w %/w %) of the total weight of the formulation.

15. The method of claim 4, wherein the administration is effective in prophylactically treating COVID-19 for 21 days to 90 days.

16. The method of claim 4, wherein the administration is effective in prophylactically treating COVID-19 for 30 days to 90 days.

17. The method of claim 4, wherein the administration is effective in prophylactically treating COVID-19 for 30 days to 60 days.

18. The method of claim 4, wherein the method is implemented in an absence of a loading dose or supplemental oral dose of ivermectin.

19. The method of claim 3, further comprising administering an oral loading dose of ivermectin prior to or immediately before or immediately after administering the diblock/triblock polymer formulation of ivermectin.

20. The method of claim 4 for implementation over a period of at least 6 months.

21. The method of claim 4, for implementation over a period of at least 15 months.

22. The method of claim 5, wherein the formulation is presented in a single prefilled syringe (PFS).

23. The method of claim 22, wherein a volume in the prefilled syringe is between 0.1 mL and 1.5 mL, preferably between 0.1 mL and 1 mL, more preferably between 0.1 mL and 0.8 mL.

24. The method of claim 5, wherein said administering is through subcutaneous administration into the subject's abdomen.

25. The method of claim 5, wherein said administering is through subcutaneous administration into the subject's upper arm.

26. The method of claim 4, wherein the formulation is excisable following administration into the subject.

27. The method of claim 5, wherein a ratio of the biodegradable triblock copolymer of (b) and the biodegradable diblock copolymer of (c) is 2:1 in said formulation.

28. The method of claim 5, wherein the pharmaceutical formulation comprises 16.25 to 25% ivermectin, 8.3% P1R4 and 4.2% dP2R3.

29. The method of claim 3, wherein the triblock copolymer has the formula:
poly(lactic acid)v-poly(ethylene glycol)w,-poly(lactic acid)x, wherein v and x are numbers of repeat units ranging from 24 to 682 and w is a number of repeat units ranging from 4 to 273 and v=x or v≠x; and
the diblock copolymer has the formula:
methoxy poly(ethylene glycol)y-poly(lactic acid)z, wherein y and z are numbers of repeat units, wherein y is a number of repeat units ranging from 3 to 45, and z is a number of units ranging from 7 to 327.
